(19) United States
(12) Patent Application Publication
(10) Pub. No.: US 2009/0088560 A1
(43) Pub. Date: Apr. 2, 2009

(54) Process for Nucleic Acid Purification

(76) Inventor: Hong Shen, San Diego, CA (US)

Correspondence Address: SUTHERLAND ASBILL & BRENNAN LLP, 999 PEACHTREE STREET, N.E., ATLANTA, GA, 30309, US

(21) Appl. No.: 12/244,374

(22) Filed: Oct. 2, 2008

Related U.S. Application Data
(60) Provisional application No. 60/976,958, filed on Oct. 2, 2007.

(51) Int. Cl. C07H 21/00; C07H 1/06

(52) U.S. Cl. 536/23.1; 536/55.3

(57) ABSTRACT

This invention provides a method of isolating and purifying nucleic acid using a binding buffer comprising a sodium- or potassium-ion-containing solution with the final concentrations of either sodium- or potassium-ion concentration of at least about 500 mM, preferably greater than about 1 M to saturate, and the pH of such solution of being adjusted in the range of about 2.0 to 5.0, for reversible binding of the nucleic acid to a silicon-containing matrix. The invention further provides a method of increasing reversible binding of the nucleic acid to a silicon-containing matrix using the binding buffer of the invention in addition to 20% to 50% (v/v) of a water-soluble organic solvent, e.g., ethanol. Nucleic acid obtained thereof that is free of chaotropes and other toxic chemicals, and nucleic acid purification kits comprising the binding buffer of the invention are also provided.

### CROSS REFERENCES TO RELATED APPLICATIONS

This application claims priority to U.S. Provisional Patent Application No. 60/976,958 filed on Oct. 2, 2007.

### FIELD OF THE INVENTION

The present invention relates to nucleic acid isolation and purification. More particularly, the present invention relates to a process and kit for isolating and purifying nucleic acids such as DNA or RNA or a hybrid molecule of DNA and RNA using a binding buffer comprising sodium- or potassium-ion and free of chaotropic salt, with or without 20-50% (v/v) of water-soluble organic solvent, e.g., ethanol, for increased reversible binding of nucleic acids to a silicon-containing matrix. Advantageously, because no chaotropic agents or other poisonous or costly agents are used in the prevent invention, the highly purified nucleic acids obtained from the present invention can be used widely, especially in food industry and pharmaceutical industry.

### BACKGROUND OF THE INVENTION

Separation and preparation of high purified target substances from different biomaterials is a very important technique because the natural biomaterials such as tissue, cell, blood, bacteria or the artificial biomaterials, such as the product of polymerase chain reaction are both complicated mixture. In research and other applications, target substances from these biomaterials are often needed to be isolated and purified. For example, natural deoxyribonucleic acid (DNA) often exists with other biosubstances, such as proteins, lipids•carbohydrates and other components in the form of mixture. The Methods for separating and purifying DNAs, such as plasmid DNA, phage DNA and chromosome DNA are of critical importance in molecular biology, pharmaceutical industry and gene therapy.

There are two kinds of DNA purification methods. One is the purification of artificially constructed DNA, such as the purification of recombined plasmid or phage from their hosts after culturing. This is one of the basic techniques used in routine molecular biology laboratory. The other one is the purification of genomic DNA from the chromosomes of eukaryotes and prokaryotes. The technique of DNA purification not only makes the research on gene functions much easier but also makes the construction of DNA library available.

In recent years a number of isolation and purification methods have been reported and commercialized. Early methods relied upon performing extended centrifugation steps or two phase extractions using aqueous phenol or chloroform plus ethanol precipitation and wash steps. These techniques are time consuming, and can require expensive instrumentation and costly and noxious reagents. Chromatographic techniques, particularly high pressure liquid chromatography and column chromatography, have been used successfully for shorter chain nucleic acids. However, longer chain nucleic acids frequently experience chain scission from excessive mechanical agitation.

Recently commercialized purification systems rely upon the ability of DNA to bind to the surface of glass and/or silicates, such as diatomaceous earth preparations or glass beads. These systems provide glass or silicate slurries for isolating DMA from heterogeneous mixtures and solutions such as low ionic strength buffers and organic solvents, for subsequently washing the immobilized DNA and silica and then eluting the DNA from the surface of the glass or silicate. A common disadvantage associated with these systems is that the isolated DNA is not sufficiently pure for many particularly demanding procedures and subsequent amplification reactions. Thus, further purification of the eluted and recovered DNA is required, which in turn adds additional time and expense to the purification process.

Other methodologies for isolating DNA from mixtures have been suggested in recent years. For example U.S. Pat. No. 5,057,426 suggests separating DNA from mixtures containing DNA by fixing the DNA onto an anion exchange resin and removing the resin from the mixture by filtration. To recover the fixed DNA from the resin, the DNA is differentially eluted using salts or other ionic systems which compete for sites on the anion exchange resin.

In a different approach, U.S. Pat. No. 4,923,978 suggests treating a solid material such as glass beads or silica so that its surface is coated with a hydrophilic material. These surfaces are said to selectively bind proteinaceous materials and not DNA. Thus, DNA can be isolated by allowing DNA and protein containing mixtures to come into contact with the treated solid material and subsequently removing the treated material, leaving behind isolated DNA which is suspended or in solution.

Characteristic of most bacterial plasmid purification methodologies is an alkaline lysis procedure, followed by treatment with silica in the presence of a chaotrope and then a wash step and an elution step. Noticeably, some silicon-containing materials can absorb target substances with the existence of so called binding agent or binding enhancer. After washing down the impurities, the purified target material can be eluted from the silicon carrier. U.S. Pat. No. 6,218,531 discloses a method for isolating RNA with silicon binding carrier in the existence of chaotropic reagents from lysed biomaterial.

The principle of such isolating methods is that the silicon-containing materials can reversibly bind DNA, RNA and hybridized molecules of DNA and RNA under the existing of binding reagents. The most important binding reagents are the chaotropic reagent (See U.S. Pat. No. 4,900,677). The common chaotropic reagents are NaI, urea, guanidine hydrochloride, NaClO4, KBr, etc. Alcohol Is also the binding reagent, such as 100% ethanol (See the background of European Pat. App. No. 0512676 A1 and U.S. Pat. No. 5,783,686).

Most of the binding reagents are toxic and harmful to human being. There are many researches on reducing the use of toxic binding reagents or not using toxic binding reagents (See U.S. Pat. No. 5,342,931; U.S. Pat. No. 5,503,816; U.S. Pat. No. 5,693,785; and U.S. Pat. No. 5,674,997). However, all these methods disclosed need special chemical modification of silicon-containing materials which increases the costs and needs special chemical equipments

Use of the traditional binding reagents, especially chaotropic reagents should be avoided or reduced in separating and purifying biomaterials, particularly DNA, by using silicon-containing materials as reversible absorbing material. Particularly, the use of chaotropic reagents or chaotropic salts is forbidden in the food and pharmaceutical industry because even trace residual of chaotropic reagents should be very harmful to human being.

Certain efforts of avoiding or reducing in use of chaotropic and other toxic and/or harmful chemicals, such as guanidine salts and sodium iodine, in the nucleic acid purification processes have been reported. For instance, U.S. Pat. No. 5,342,931 discloses a hydroxylated silica polymer, other than the chaotropic reagents or other toxic chemicals, as DNA binding carrier while the impurities were washed away; U.S. Pat. No. 5,503,816 discloses a method of binding the DNA to chemically modified silicon-containing materials with sufficient hydrophilicity and electropositivity without the use of chaotropic reagents; U.S. Pat. No. 6,855,499 describes a method that uses salt and polyalkylene glycol as binding buffer for DNA to bind to magnetizable cellulose or its derivatives; U.S. Pat. No. 6,433,160 discloses an acidic solution without the chaotropic reagents as DNA binding buffer for the reversible binding of a nucleic acid molecule to paramagnetic particles; U.S. Pat. No. 5,972,613 discloses a method for enrichment of RNA with respect to DNA by forming a RNA-containing precipitate and the RNA is then isolated form DNA by centrifugation; and U.S. Pat. No. 6,291,248 discloses the use of modified silicon carbide as the binding carrier with 2 M potassium acetate without the use of chatropic salts.

Water soluble organic solvents have also been used to replace the chaotropic reagents and other toxic chemicals as binding reagents in the nucleic acid purification process. For instance, European Patent Application No. 0 512 767 A1 and U.S. Pat. No. 5,783,686 disclose the use of water soluble organic solvents, such as ethanol, isoproponal, or 100% ethyl alcohol as a binding agents to replace chaotropes to facilitate binding DNA to reversibly bind to the surface of silicon-containing particles; U.S. patent application Ser. No. 10/555,798 (abandoned) and the EP Patent Application No. 04717542.7 disclose an acidic potassium-containing solution, with a pH value from 2 to 4, as binding buffer for reversible binding of nucleic acid molecule to silicon-containing materials.

Nucleic acid purification or isolation is the fundamental and critical procedure in the field of molecular biology. With the rapid advances in biomedical, biopharmaceutical, and bioagricultural research, there is a need for the continued improvement of nucleic acid purification technology with respect to simplify the purification procedure, obtain high yield and high purity product, reduce costs for expensive equipments and reagents, avoid using chaotropic and/or other toxic reagents, and improve safety for researchers and the environment.

### SUMMARY OF THE INVENTION

The present invention provides a method of isolating and purifying nucleic acid from nucleic acid-containing biological samples using a binding buffer comprising a sodium- or potassium-ion-containing and chaotropic salts free solution for reversible binding of the nucleic acid to a silicon-containing matrix. The present invention further provides a method of isolating and purifying nucleic acid from nucleic acid-containing biological samples using the binding buffer of the invention in combination with a water-soluble organic solvent to increase reversible binding of the nucleic acid to the silicon-containing matrix.

In one embodiment of the invention, a sodium-ion-containing and chaotropic salts free aqueous solution is used as the binding buffer for reversible binding of nucleic acid to a silicon-containing matrix. The solution consists of sodium ions with the final concentration in the range of about 1.0 M to saturation. In yet one embodiment, the final concentration of sodium ions in the solution is about 1.0 M to about 3.0 M. The pH of the solution is adjusted in the range of about 2.0 to 5.0 by acidic acids or other acids. In yet one embodiment, the pH of the solution is adjusted to equal to or less than about 4.8. The sodium ions in the solution are derived from any salts include, but not limited to, sodium acetate, sodium chloride, sodium citric, and sodium phosphate.

In yet one embodiment of the invention, the sodium-ion-containing aqueous solution of the invention is referred to as buffer S1. One example of buffer S1 contains about 2.0 M to 2.5 M sodium acetate with a pH of about 4.0 to 4.8.

The invention further provides a method of isolating and purifying nucleic acid from nucleic acid-containing biological samples using the sodium-ion-containing and chaotropic salts free binding solution in combination with a water-soluble organic solvent. In one embodiment of the invention, the water-soluble organic solvent includes, but is not limited, to ethanol, isopropanol, methanol, ethyl alcohol, or the like. In yet one embodiment, the water-soluble organic solvent is ethanol. In yet another embodiment of the invention, the final concentration of the water-soluble organic solvent is about 20% to 50% (v/v). In yet another embodiment of the invention, the final concentration of the water-soluble organic solvent is about 20% to 30% (v/v). The invention provides that addition of the water-soluble organic solvent, e.g., ethanol, in the binding buffer of the invention significantly increases the reverse binding of nucleic acid to the silicon-containing matrix as compared to use the binding buffer of the invention and/or water-soluble organic solvents alone.

The invention also provides a potassium-ion-containing and chaotropic salts free aqueous solution used as the binding buffer for reversible binding of nucleic acid to a silicon-containing matrix. The solution consists of potassium ions with the final concentration at least about 500 mM. The pH of the potassium-ion-containing solution is adjusted in the range of about 4.0 to 5.0 by acetic acids or other acids. The invention further provides that the potassium-ion-containing binding solution is used in combination of about 20 to 30% (v/v) of water-soluble organic solvent, including, but not limited to ethanol, isopropanol, methanol, or the like, for increased reversible binding of nucleic acid to the silicon-containing matrix.

The invention further provides a kit for purifying or isolating nucleic acid from any nucleic acid containing biological samples and/or enzymatic reactions, such as PCR, restriction digestion, nick translation, labeling, sequencing and tailing. The kit comprises the sodium- and/or potassium-ion-containing binding buffer of the invention and an operating protocol and/or instruction for the isolation and purification methods. In one embodiment of the invention, the kit provides methods and buffers for the purification or isolation procedure. Such methods comprise the steps of first combining an appropriate amount of the binding buffer of the invention comprising a sodium- or potassium-ion-containing solution with the samples in which the target nucleic acids are contained, and further apply the mixture to a silicon-containing matrix. The binding buffer of the invention creates strong binding of nucleic acids to any silicon-containing matrix. The kit of the invention also provides wash buffer to be used to wash out other unwanted impurities from the matrix. The target nucleic acids are then eluted with TE buffer or deinoized H₂O from the matrix. Furthermore, the kit of the invention also provides an operating protocol and/or instruction of using about 20% to 50% of a water-soluble organic solvent, such as ethanol, in combination with the binding buffer for increased reversible binding of nucleic acid to the silicon-containing matrix.

In yet another embodiment, a kit for purification or isolation of plasmid DNA is provided. Such methods include the steps of lysing the transformed bacterial, neutralizing the lysate with the binding buffer of the invention comprising a potassium or sodium-ion-containing solution, clearing the lysate by centrifugation or filtration, mixing the cleared lysate with about 20% to 30% (v/v) ethanol, and loading the mixture to a silicon-containing matrix. The combination of the potassium or sodium-ion-containing binding buffer with about 20% to 30% (v/v) ethanol creates strong reversible binding of nucleic acids to any silicon-containing matrix. The other unwanted impurities are washed out by a wash buffer. The target nucleic acids are then eluted with TE buffer or deinoized H₂O from the matrix.

Nucleic acids isolated and purified using the binding buffer of the present invention, and methods of using thereof, are also provides. The isolated and purified nucleic acids obtained from the present invention are free of chaotropes or other toxic chemicals, and are suitable for a wide range of downstream applications, including, but not limited to gene therapy, genetic vaccination, and other enzymatic reactions selected from the group consisting of PCR, restriction digestion, nick translation, labeling, sequencing and tailing. The present invention also provides that the nucleic acid isolation and purification methods disclosed herewith can be used for isolating and/or purifying any nucleic acids, including, but not limited to DNA (e.g., plasmid DNA), RNA, or a hybrid molecule of DNA and RNA, from any nucleic acid-containing biological samples. Examples of such biological samples include, but are not limited to animal or human bodily fluids (e.g., whole blood, blood serum, urine, feces, liquor cerebrospinalis, sperm, saliva), tissues, organs, cells, cell cultures, PCR products, and the like.

### BRIEF DESCRIPTION OF THE DRAWINGS

FIG. 1 illustrates an effect of sodium ion concentration on nucleic acid binding. One uL of eluted DNA mixed with 1 uL of 10× loading buffer and 8 uL of deionized H₂O and loaded into 1% agarose gel. The DNAs were visualized by UV Transilluminator with ethdium bromide stain. Lane 1 to 6 corresponds to the numbers in Table I.

FIG. 2 illustrates an effect of pH of the sodium-ion-containing solution on nucleic acid binding. One uL of eluted DNA mixed with 1 uL of 10× loading buffer and 8 uL of deionized H₂O and loaded into 1% Agarose gel. The DNAs were visualized by UV Transilluminator with ethdium bromide stain. Lane 1 to 6 corresponds to the sample number in Table II.

FIG. 3 illustrates that combination of sodium acetate solution with ethanol ((50% v/v) significantly increase the binding of nucleic acids to the silicon-containing matrix. One uL of eluted DNA mixed with 1 uL of 10× loading buffer and 8 uL of deionized H₂O and loaded into 1% Agarose gel. The DNAs were visualized by UV Transilluminator with ethdium bromide stain. Lane 1 to 6 corresponds to the numbers in Table III.

FIG. 4 illustrates plasmid purification or isolation using binding buffer N1 comprising 3M potassium acetate solution (pH 4.8) and 20% to 30% (v/v) ethanol. One uL of eluted DNA mixed with 1 uL of 10×. loading buffer and 8 uL of deionized H₂O and loaded into 1% Agarose gel. The DNAs were visualized by UV Transilluminator with ethdium bromide stain. Lane 1 to 6 corresponds to the numbers in Table IV.

### DETAIL DESCRIPTION OF THE INVENTION

The present invention provides a method of isolating and purifying nucleic acid from nucleic acid-containing biological samples using a binding buffer comprising a sodium- or potassium-ion-containing and chaotropic salts free solution for reversible binding of the nucleic acid to a silicon-containing matrix. The present invention further provides a method of isolating and purifying nucleic acid from nucleic acid-containing biological samples using the binding buffer of the invention in combination with a water-soluble organic solvent to increase reversible binding of the nucleic acid to the silicon-containing matrix

There are two major nucleic acid purification systems, ion exchange based and silicon based. Silicon-containing material possess the reversible association and/or dissociation characteristics that are widely used in the nucleic acid purification or isolation procedure. In general, the binding of nucleic acids to silica materials, such as silica, celite, glass powders and the like, needs the existence of high concentration of chaotropic reagents or alcohol. To avoid the many disadvantages of using chaotropic reagents and alcohol as binding reagents, some new methods have been developed. However, all these methods increase the difficulty of procedure and restrict the utilizing of binding carriers. The binding solution of present invention not only avoids using the traditional harmful binding reagents but also can be used to different silicon-containing materials. Because the binding solution is nontoxic, inexpensive, and easy to prepare and handle, it can be a good substitute of the traditional binding reagents.

The present invention provides a binding solution that is an acidic aqueous solution containing sodium ion. The sodium-ion containing binding solution of the present invention is free of any chaotropes or other toxic chemicals, including but not limited to guanidine salts or sodium iodine. The final concentration of the sodium ion in the solution is in the range of about 1.0 M to saturation, or about 1.0 M to about 3.0 M. The pH of the sodium-ion-containing solution is adjusted in the range of about 2.0 to 5.0, or equal to or less than about 4.8, by acidic acids or other acids. The sodium ions in the solution are derived from any salts include, but not limited to, sodium acetate, sodium chloride, sodium citric, and sodium phosphate. A mixture of different sodium salts can also be used in the solution.

In one embodiment, the sodium-ion-containing binding solution of the present invention contains sodium acetate (NaAc) in a concentration of about 1.0M, 1.5M, 2.0 M, 2.1 M, 2.2 M, 2.3 M, 2.5M, 2.7M, 2.8M, 3.0M, 3.2M, 3.4M, 3.6M, 3.8M, or 4.0M. In yet another embodiment, the sodium-ion-containing binding solution of the present invention has a pH adjusted to 2.0, 2.5, 3.0, 3.1, 3.3, 3.5, 3.7, 3.9, 4.1, 4.3, 4.5, 4.8. In yet another embodiment, the strong nucleic acid binding is obtained using a sodium acetate (2.7M) with a pH of equal to or less than 4.8. In yet another embodiment. In one embodiment of the invention, a sodium-ion-containing and chaotropic salts free aqueous solution is provided and used as the binding buffer for reversible binding of nucleic acid to a silicon-containing matrix. In yet one embodiment of the invention, binding buffer S1 is provided. One example of binding buffer S1 contains about 2.0 M to 2.5 M sodium acetate with a pH of about 4.0 to 4.8.

The present invention further provides a binding solution that is an acidic aqueous solution containing potassium ion. The potassium-ion-containing binding solution of the present invention is free of any chaotropes or other toxic chemicals, including but not limited to guanidine salts or sodium iodine. The final concentration of the potassium ion in the solution is at least 500 mM. The pH of the potassium-ion-containing solution is adjusted in the range of about 4.0 to 5.0 by acidic acids or other acids. The potassium ion can be obtained from KCl, K₂SO₄ [symbol] KNO₃, or potassium acetate. A mixture of different potassium salts can also be used in the solution such as the mixture of KCl and K₂SO₄. In one embodiment, potassium acetate (3M, pH 4.8) is used in the present invention.

Because of the difference of the solubility of various sodium or potassium salts in water, the upper limit of the concentration of either sodium or potassium salts in present invention is the saturation. The solubility of sodium or potassium salts solution is changed with the temperature. The saturated concentration of sodium or potassium salt is under room temperature, but it does not mean that the present invention can not be applied under other temperature. In fact, the present invention can be used at different temperature. Because the saturated concentration of either sodium or potassium salts changes with temperature, the changes of saturated concentration of sodium or potassium ion under different temperature are included in the scope of the present invention.

Sodium or Potassium ion comes from the dissolving of potassium salts in water. Because sodium or potassium ion can come from resolving of more than one sodium or potassium salt in solution, the solute in the solution of present invention can be either one sodium or potassium salt, or the mixture of several sodium or potassium salts. The concentration of the sodium or potassium ion in the solution is the sum of sodium or potassium ion from all dissolved potassium salts. The concentration of sodium ion is about 1M to saturation, and the potassium ion is at least 500 mM or up to the saturated concentration. As mentioned above, the temperature can be room temperature or any higher temperature. Thus, the saturation of sodium or potassium salts will change with the change of temperature.

As used herein, acids are used to adjust the acidity of the solution. The available acids comprising weak acids and strong acids include, but not limited to acetic acid, sulfuric acid, nitric acid and phosphoric acid. In one embodiment, weak acids such as acetic acid, propionic acid are used. In yet another embodiment, the strong acids such as hydrochloric acid, sulfuric acid, nitric acid and phosphoric acid can also be used. Because it is more convenient to use weak acids to adjust the pH of the binding solution, weak acid, such as acetic acid, is more often used.

The invention further provides a method of isolating and purifying nucleic acid from nucleic acid-containing biological samples using the sodium-ion-containing and chaotropic salts free binding solution in combination with a water-soluble organic solvent. In one embodiment of the invention, the water-soluble organic solvent includes, but is not limited, to ethanol, isopropanol, propanol, methanol, ethyl alcohol, or the like. In yet one embodiment, the water-soluble organic solvent is ethanol. In yet another embodiment of the invention, the final concentration of the water-soluble organic solvent is about 20% to 50% (v/v). In yet another embodiment of the invention, the final concentration of the water-soluble organic solvent is about 20% to 30% (v/v). The invention provides that addition of the water-soluble organic solvent, e.g., ethanol, in the binding buffer of the invention significantly increases the reverse binding of nucleic acid to the silicon-containing matrix as compared to use the binding buffer of the invention and/or water-soluble organic solvents alone.

The invention further provides a buffer N1 which comprises a potassium-ion-containing binding solution, e.g., 3M potassium acetate, pH 4.8, in combination with about 20 to 30% (v/v) of water-soluble organic solvent, e.g., ethanol. The buffer N1 of the present invention can be used as both the neutralization buffer and the binding buffer for increased reversible binding of nucleic acid to the silicon-containing matrix.

As used herein, any silicon-containing materials including unmodified, modified or specially-prepared can be used in the present invention. It includes, but is not limited to, silica, glass celite, and the like. The material can be in different forms which have sufficient surface to bind target materials, such as DNA. For example, the glass can be powder or fiber. The silicon-containing material such as glass powder, glass fiber or celite which exhibits sufficient hydrophilicity and sufficient electropositivity are often used. The special silicon materials disclosed in the U.S. Pat. Nos. 5,342,931♦5,503,816♦5,693,785 and 5,674,997 certainly can be used in the present invent. In one embodiment of the present invention, the silicon-containing material can be in the form of powder, which can be suspended in solution and absorb the target biological substance such as DNA. The silicon-containing material can also be filled into a column and the target biomaterial can be separated and purified by the column.

The present invention provides that a chaotropic salt free binding buffer comprising either sodium- or potassium ion-containing acid solution, alone and/or particularly in combination with a water-solution organic solvent, e.g., ethanol, significantly enhances the reversible binding of target biomaterials especially nucleic acids (e.g., DNA, RNA, or a hybrids of DNA and RNA) to different silicon-containing materials. High efficiency and high purity of the target nucleic acids can be achieved. The purified nucleic acids free of chaotropes or other toxic chemicals, and can then be used in many fields, especially in pharmaceutical industry.

As used herein, a purified nucleic acid is intended to refer to a composition, isolatable from other components, wherein the target nucleic acid is purified to any degree relative to its naturally-obtainable state. An isolated or purified nucleic acid also refers to a nucleic acid free from the environment in which it may naturally occur. Generally, “purified” refers to a nucleic acid composition that has been subjected to fractionation to remove various other components, and which composition substantially retains its expressed biological activity. Where the term “substantially purified” is used, this designation refers to a composition in which the target nucleic acid forms the major component of the composition, such as constituting about 50%, about 60%, about 70%, about 80%, about 90%, about 95%, or more of the target nucleic acid in the composition.

As used herein, the target biomaterials can be protein, nucleic acids, and the like. As used herein, the “nucleic acids/nucleotides” may be derived from genomic DNA, complementary DNA (cDNA) or synthetic DNA. The term “nucleic acid/nucleotide” also refers to RNA or DNA that is linear or branched, single or double stranded, chemically modified, or a RNA/DNA hybrid thereof. In one embodiment, the present invention is used in isolating and/or purifying DNA from any biological sample. As used herein, the biological samples include, but are not limited to animal or human bodily fluids (e.g., whole blood, blood serum, urine, feces, liquor cerebrospinalis, sperm, saliva), tissues, organs, cells, cell cultures, PCR products, and the like,

As used herein, an “isolated” nucleic acid molecule is one that is substantially separated from other nucleic acid molecules which are present in the natural source of the nucleic acid (i.e., other sequences encoding other polypeptides). An “isolated” nucleic acid is free of some of the sequences which naturally flank the nucleic-acid (i.e., sequences located at the 5′ and 3′ ends of the nucleic acid) in its naturally occurring replicon. For example, a cloned nucleic acid is considered isolated. A nucleic acid is also considered isolated if it has been altered by human intervention, or placed in a locus or location that is not its natural site, or if it is introduced into a cell by agroinfection. Moreover, an “isolated” nucleic acid molecule, such as a cDNA molecule, can be free from some of the other cellular material with which it is naturally associated, or culture medium when produced by recombinant techniques, or chemical precursors or other chemicals when chemically synthesized.

Therefore, the present invention provides a novel sodium-ion-containing binding solution for nucleic acid purification or isolation. The final concentration of the sodium ion in the binding solution is at least 1.0 M or up to saturation with the pH in the range of about 2.5 to about 5.0. In one embodiment, the present invention provides that the sodium-ion-containing binding solution is first mix with a biological sample, which contains the target nucleic acid; the mixture is then loaded into a silicon-containing matrix. While target nucleic acid binds to the matrix, unwanted impurities such as proteins and salts are washed out by a wash buffer. The target nucleic acid is then eluted by TE buffer or deionized water. The present invention is suitable for purifying or isolating DNA and RNA from enzymatic reactions such as PCR, restriction digestion, nick translation, labeling, sequencing, desalting and tailing.

In yet another embodiment, this invention further proves that a combination of the sodium-ion-containing binding solution with a water-soluble organic solvent for increased reversible binding of nucleic acid to a silicon-containing matrix. Although the EP Patent No. 0512676 A1 and U.S. Pat. No. 5,783,686 discloses the use of water-soluble organic solvents as binding reagents without the addition of ions such potassium ions or sodium ions, the present invention provides that DNA binding in the water-soluble organic solvent alone, without a combination with a sodium- or potassium-ion containing solution is quite poor. For instance, in one embodiment, the present invention provides that 10 ug of DNA was poorly binding to a silicon containing matrix in a binding solution comprising a combination of 50 uL TE buffer and 50 uL ethanol, while the mixture of 10 ug DNA in 50 uL 2.7 M sodium acetate and 50 uL of ethanol has over 87% DNA recovery from the silicon containing matrix. As used herein, the water-soluble organic solvents include ethanol, isopropanol, propanol, and methanol.

In yet another embodiment, the present invention provides an aqueous binding solution comprising 1) either potassium or sodium ions with the final concentration of at least 500 mM, 1M, or up to saturation, at the pH range of about 2.0-5.0, about 4.0 to 5.5, or equal to or less than 4.8, and 2) about 20% to about 30% or about 20% to about 50% (final percentage by volume) water-soluble organic solvent, such as ethanol. The aqueous binding solution of the present invention is used as binding buffer for, but not limited to, plasmid DNA purification or isolation. In one embodiment, the present invention provides a buffer N1 solution, which comprises a potassium or sodium-ion-containing aqueous solution plus about 20% to 30% of ethanol. The buffer N1 solution is optimized for, but not limited to, plasmid DNA purification or isolation in which the pH after neutralization is adjusted for pure plasmid yield without RNA contamination.

The present invention further provides that in the process of plasmid DNA purification, after neutralization, the pH range of 4 to 6 has significant advantage over the pH range of 2 to 4 to break down RNA. In addition, with the addition of about 20% to about 30% of ethanol by volume, the yield of the plasmid DNA increases at least 2-fold as compared to the yield produced by buffer C1 disclosed in the U.S. patent application Ser. No. 10/555,798 and BP Patent Application No. 04717542.7. The present invention provides that the buffer N1 solution disclosed herewith has superior reversible binding ability for nucleic acid to silicon-containing matrix as compared to buffer C1 disclosed in the U.S. patent application Ser. No. 10/555,798 and EP Patent Application No. 04717542.7.

The invention further provides a kit for purifying or isolating nucleic acid from any nucleic acid containing biological samples and/or enzymatic reactions, such as PCR, restriction digestion, nick translation, labeling, sequencing and tailing. The kit comprises the sodium- and/or potassium-ion-containing binding buffer of the invention and an operating protocol and/or instruction for the isolation and purification methods. In one embodiment of the invention, the kit provides methods and buffers for the purification or isolation procedure. Such methods comprise the steps of first combining an appropriate amount of the binding buffer of the invention comprising a sodium- or potassium-ion-containing solution with the samples in which the target nucleic acids are contained, and further apply the mixture to a silicon-containing matrix. The binding buffer of the invention creates strong binding of nucleic acids to any silicon-containing matrix. The kit of the invention also provides wash buffer to be used to wash out other unwanted impurities from the matrix. The target nucleic acids are then eluted with TE buffer or deinoized H₂O from the matrix. Furthermore, the kit of the invention also provides an operating protocol and/or instruction of using about 20% to 50% of a water-soluble organic solvent, such as ethanol, in combination with the binding buffer for increased reversible binding of nucleic acid to the silicon-containing matrix.

In yet another embodiment, a kit for purification or isolation of plasmid DNA is provided. Such methods include the steps of lysing the transformed bacterial, neutralizing the lysate with the binding buffer of the invention comprising a potassium or sodium-ion-containing solution, clearing the lysate by centrifugation or filtration, mixing the cleared lysate with about 20% to 30% (v/v) ethanol, and loading the mixture to a silicon-containing matrix. The combination of the potassium or sodium-ion-containing binding buffer with about 20% to 30% (v/v) ethanol creates strong reversible binding of nucleic acids to any silicon-containing matrix. The other unwanted impurities are washed out by a wash buffer. The target nucleic acids are then eluted with TE buffer or deinoized H₂O from the matrix.

### EXAMPLES

The invention is further illustrated by the flowing examples, which are not to be construed in any way as imposing limitations upon the scope thereof. It is apparent for skill artisans that various modifications and changes are possible and are contemplated within the scope of the current invention.

### Example I

### The Effect of Sodium Ion Concentration on Nucleic Acid Binding

In this experiment, the sodium ion-containing aqueous solution is proved to be a novel binding buffer for reversible binding of nucleic acid to silicon-containing matrix without any chaotropic reagents or other guanidine salts. The sodium ions used in this experiment is sodium acetate trihydrate (Sigma catalog #S7670), the pH is adjusted by acetic acids. The silicon-containing column is manufactured by Qiagen (cat #28104). In this experiment, the effect of sodium ion concentration on DNA binding was tested. The procedure for this experiment was carried as follows:
1. Mix 90 uL buffer S1 with 10 uL of DNA (pcDNA3 based vector, in TE buffer). Buffer S1: 2.7 M sodium acetate, pH 4.3.
2. Load the mixture to a mini column
3. Centrifuge at 13,000 rmp for 1 minute.
4. Wash the column with 500 uL DNA wash buffer (20 mM Tris-HCL, pH 7.5, 20 mM EDTA, 80% ethanol). Decant and repeat once.
5. Centrifuge the empty column for 1 minute at 13,000 rpm,
6. Put the column into a clean 1.5 mL, centrifuge tube and add 50 uL of TE buffer, incubate at room temperature for 1 minute.
7. Centrifuge at 13,000 rpm to elute the DNA.
8. Check the DNA concentration by OD260/280 and load 1 uL of sample on 1% agarose gel.

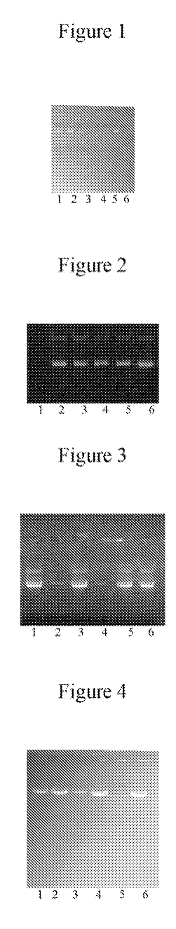

The results presented in Table 1, below and FIG. 1. indicate that sodium-ion-containing solution is a novel non-chaotropic binding buffer for reversible binding of nucleic acids to silicon-containing matrix. The binding ability decreases with the deduction of sodium ion concentration in the solution. At final concentration of 1.89 M (No. 2), the DNA recovery is greater than 90% while there is only 21.8% DNA recovery at 0.81 M (No. 4). The concentration of the sodium ions is important for the binding of nucleic acid to the silicon-containing matrix. The results from samples No. 1, No. 2 and No. 6 also indicate that the sodium-ion-containing solution has equivalent binding ability as compared to the binding solution comprising a chaotropic reagent.

TABLE I

| Sample | Buffer | DNA | Buffer | TE | Final NaAc | DNA yield | DNA |
|---|---|---|---|---|---|---|---|
| No. | S1* (uL) | (uL) | PBT** (uL) | (uL) | Concentration | ug | recovery % |
| 1 | 90 | 10 | | 0 | 2.43 M | 9.07 | 90.7 |
| 2 | 70 | 10 | | 20 | 1.89 M | 9.04 | 90.4 |
| 3 | 50 | 10 | | 40 | 1.35 M | 4.15 | 41.5 |
| 4 | 30 | 10 | | 60 | 0.81 M | 2.18 | 21.8 |
| 5 | 10 | 10 | | 80 | 0.27 M | <1 | <10% |
| 6 | 0 | 10 | 50 | 40 | | 9.05 | 90.5 |

*Buffer S1: 2.7 M NaAc, pH4.3 adjusted by acedic acid.
DNA: pDNAs vector at 1 ug/uL in TE buffer.
**Buffer PBT: contains chaotropic salt. (Qiagen PCR extraction kit cat# 28104)

### Example II

### The Effect of pH of the Sodium-Ion-Containing Solution on Nucleic Acid Binding

In this experiment, the effect of pH of the sodium-ion-containing binding solution on nucleic acid binding was tested and the optimized pH range was found for strong nucleic acid binding. The procedure was carried out as follows,
1. Prepare 2.3 M NaAc (Buffer S1) at six different pH values: 5.2, 4.8, 4,5, 4.3, 4.1 and 3.9. The pH is adjusted by acedic acid.
2. Mix 10 uL of DNA (pcDN A3.1) with 90 uL, of buffer S1 (buffer S1 at 6 different pH).
3. Load the mixture to the silicon-containing mini column.
4. Centrifuge at 13,000 rmp for 1 minute.
5. Wash the column with 500 uL DNA wash buffer (20 mM Tris-HCL, pH 7.5, 20 mM EDTA, 80% ethanol). Decant and repeat once.
6. Centrifuge the empty column for 1 minute.
7. Put the column into a clean 1.5 mL centrifuge tube and add 50 uL of TE buffer and incubate at room temperature for 1 minute.
8. Centrifuge to elute the DNA.
9. Check the DNA concentration by OD260/280 and load 1 uL of sample on 1% agarose gel.

The results presented in Table II and FIG. 2 showed that the effective pH range for strong nucleic acid binding is about 4.8 and below.

TABLE II
The effect of pH on nucleic acid binding

| | | | DNA yield | % |
|---|---|---|---|---|
| | Buffer S1* | 10 ug DNA (uL) | ug | of DNA recovery |
| 1 | 10 uL pH 5.2 | 10 | <1 | <10% |
| 2 | 10 uL pH 4.8 | 10 | 8.88 | 88.8 |
| 3 | 10 uL pH 4.5 | 10 | 9.17 | 97.7 |
| 4 | 10 uL pH 4.3 | 10 | 9.22 | 92.2 |
| 5 | 10 uL pH 4.1 | 10 | 8.89 | 88.9 |
| 6 | 10 uL pH 3.9 | 10 | 9.24 | 92.4 |

*Buffer S1: 2.3 M NaAc at 6 different pHs.
DNA: 1 ug/uL in TE buffer
DNA concentration is evaluated by OD260.

### Example III

### Increased Bin Din a of Nucleic Acid to Silicon-Containing Matrix in a Combination of a Potassium or Sodium-Ion-Containing Solution with 50% (v/v) Ethanol

The addition of potassium or sodium-ion-containing solution to water-soluble organic solvents such as ethanol significantly increased the binding of nucleic acids to a silicon-containing matrix. The procedure was carried out as follows,
1. Mix 10 uL of DNA solution (in TE buffer) with buffer S1 (2.7M NaAc, pH 4.3)
2. Add TE buffer or ethanol (see table III).
3. Load the sample to a mini column.
4. Centrifuge at 13000 rpm for 1 minute. Decant.
5. Wash with 500 uL of DNA wash buffer (20 mM Tris-HCL, pH 7.5, 20 mM EDTA, 80% ethanol). Repeat once.
6. Centrifuge the empty column for 1 minute at 13,000 rpm.
7. Add 60 uL of TE buffer to the center of the column, incubate at room temperature for 1 minute.
8. Centrifuge at 13,000 rpm to elute the DNA.
9. Check DNA concentration by OD260/280 and load 1 uL of sample to 1% TAE agarose gel.

The results presented in Table III and FIG. 3 indicate that the addition of sodium-ion-containing solution to the ethanol significantly increased the reversible binding of nucleic acids to a silicon-containing column (No. 3). This study indicates that the high sodium ion concentration favors the binding of nucleic acid to the silicon-containing matix (No. 1); the low sodium salts (less than 1.21 M in final solution) does not favor the binding of nucleic acid to the silicon-containing matrix. (No. 4); DNA was poorly bound to and recovered from a silicon-containing matrix in a solution comprising a combination of 50% (v/v) ethanol and 50 uL of TE buffer without any potassium or sodium ions in the solution (No. 2); the mixture of 1.21 M sodium ions and 50% (v/v) of ethanol creates a strong binding condition for nucleic acid to a silicon-containing matrix (No. 3); the addition of sodium, potassium or other salts to the organic solvent at certain concentration provides superior binding condition for nucleic acid to a silicon-containing matrix (No. 3); the guanidine and chaotropic reagent free salt solution described in the present invention provides equivalent or better DNA recovery as compared to the traditional methods using guanidine or chaotropic reagents as binding reagents (No. 1, 3, 5 and 6).

TABLE III

| | | | TE | 100% | DNA | % of DNA |
|---|---|---|---|---|---|---|
| No. | DNA | Binding buffer | buffer | ethanol | yield | recovery |
| 1 | 10 uL | 90 uL 2.7 M buffer | | | 5.25 | 87.5 |
| | | S1 pH 4.3 | | | | |
| 2 | 10 uL | | 45 uL | 45 uL | <1 ug | <16 |
| 3 | 10 uL | 45 uL 2.7 M buffer | | 45 uL | 5.24 | 87.3 |
| | | S1 pH 4.3 | | | | |
| 4 | 10 uL | 45 uL 2.7M buffer | 45 uL | | <1 ug | <16 |
| | | S1, pH 4.3 | | | | |
| 5 | 10 uL | 90 uL of 3 M | | | | 85.0 |
| | | Guanidine-HCL | | | | |
| 6 | 10 uL | 90 uL of Qiagen's | | | | 82.5 |
| | | buffer PBT | | | | |

DNA: 6 ug pCNDAs at 0.6 ug/uL.
Qiagen's buffer PBT contains chaotropic reagent.

### Example IV

### Plasmid Purification or Isolation Using Buffer N1 as Binding Buffer

Various protocols can be followed for the purification of plasmid DMA using buffer N1 described herewith in this invention as both neutralizing and/or binding buffer. The composition of the buffers used in the experiments were as set out below:
Buffer A (Cell resuspension buffer): 50 mM Tris-HCL, pH 7.5, 10 mM EDTA, and 100 ug/mL RNAse A
Buffer B (Cell lysis buffer): 0.2M NaOH and 1% SDS (sodium dodecyl sulfate).
Buffer N1: 3 M Potassium acetate, pH 4.8. After neutralization, plus 20 to 30% by volume of water-soluble organic solvents such ethanol. Buffer N1 is both the neutralization buffer and the binding buffer.
Wash buffer: 20 mM Tris-HCL, pH 7.5, 20 mM EDTA, 80% EtOH (ethanol)
TE buffer (Elution buffer): 10 mM Tris HCL, 1 mM EDTA, pH 8.0.

The procedure was carried out as follows:
1. Pellet 1.5 mL overnight culture of Top 10 (E. Coli strain, transformed with pcDNA3.1) at 13,000 rpm for 1 minute. Discard the supernatant.
2. Resuspend the pellet with 200 uL of buffer A.
3. Add 200 uL of buffer B and mix by inverting the tube 5 times. Incubate at room temperature for 3 to 5 minutes.
4. Add 250 uL of either buffer N1 or buffer C1 described in U.S. patent application Ser. No. 10/555,798 and EP Patent Application No. 04717542.7. Mix well by inverting the tube 5 times.
5. Centrifuge the lysate at 13,000 RPM for 10 minutes.
6. Transfer the supernatant to a mini spin column with a 2 ml collection tube.
7. At this point, for sample #2, 4 and 6, add 200 uL of 100% EtOH and mix well by pipetting or inverting the columns.
8. Centrifuge at 13,000 RPM for 1 minute. Discard the flowthrough.
9. Add 500 uL wash buffer to the column and centrifuge at 13,000 RPM for 1 minute. Discard the flowthrough Repeat once.
10. Spin the empty column for 1 minute.
11. Elution the DNA with 100 uL of TE buffer by centrifugation at 13,000 rpm for 1 minute.
12. The DNA yield is then checked by OD260 and agarose gel electrophoresis.

The results from table IV and FIG. 5 provided that: a) for sample 1 and 2, the yields estimated by OD₂₆₀ were not in accordance with the yield estimated on agarose gel. The agarose gel, however, provided more accurate estimate if the DNA is contaminated with RNAs; b) the pH of buffer C1 was 1.9 and the pH after neutralization was about 3.5 as described in U.S. patent application Ser. No. 10/555,798 and EP Patent Application No. 04717542.7. Since the RNase A does not work effectively at such low pH (the working pH is at least 5 to 6), the chromosomal RNAs are not broken down effectively by RNAse A into nucleotides and could not be washed out by wash buffer, instead, the RNAs are co-purified with the plasmid DNA. The small pieces of RNAs may not be visible on agarose gel; c) the yield of the plasmid DNA was at least doubled with the addition of 20-30% ethanol or other water-soluble solvents as indicated in FIG. 4 (sample 1 and 2, sample 3 and 4, and sample 5 and 6); d) for sample 3 and 4, the yields estimated by OD₂₆₀ and agarose gel were consistent, indicating that the purified plasmid DNA was free of RNA contamination (or the RNA in the purified plasmid DNA was not significant); and e) the pH range of about 4.1 to 5.5 (preferred pH of 4.8 to 5.0) allowed RNase A to chop off unwanted RNA completely and the purity of the plasmid DNA was thus secured.

TABLE IV
Comparison study of using buffer C1 and buffer N1 as binding
buffer for plasmid DNA isolation or purification

| | | DNA yield | DNA yield | |
|---|---|---|---|---|
| Sample | | by OD260 | estimated by | |
| No | Binding buffer | (ug) | agarose gel (ug) | Notes |
| 1 | Buffer C1, pH 1.90 | 15.2 | 8.0 | RNA |
| | | | | contamination |
| 2 | Buffer C1, pH 1.90, + 200 uL | 23.6 | 16.0 | RNA |
| | EtOH | | | contamination |
| 3 | Buffer N1 (2.5 M KAc, pH 4.8) | 5.0 | 5.0 | |
| 4 | Buffer N1 (2.5 M KAc, pH 4.8) + | 21.5 | 20 | |
| | 200 uL EtOH | | | |
| 5 | Buffer N1 (2.7 M NaAc, pH 4.3) | 1.0 | 1.0 | |
| 6 | Buffer N1 (2.7 M NaAc, pH 4.3) + | 20.8 | 21.2 | |
| | 200 uL EtOH | | | |

What is claimed:

1. A method of isolating and purifying nucleic acid from a biological sample comprising providing a binding buffer comprising a sodium-ion-containing solution for reversible binding of said nucleic acid to a silicon-containing matrix, wherein said sodium-ion-containing solution is chaotropic salts free binding solution, wherein a final sodium ion concentration is greater than about 1 M to saturate, and wherein a pH of said sodium-ion-containing solution is adjusted in the range of about 2.0 to 5.0.

2. The method of claim 1, wherein said sodium-ion-containing solution comprises one or more sodium salts selected from the group consisting of sodium acetate, sodium chloride, sodium citric, and sodium phosphate.

3. The method of claim 2, wherein said sodium-ion-containing solution comprises sodium acetate.

4. The method of claim 1, wherein said final sodium ion concentration in said sodium-ion-containing solution is greater than about 1.0 M to about 3.0 M.

5. The method of claim 1, wherein said pH of said sodium-ion-containing solution is adjusted by acetic acid or other acids.

6. The method of claim 5, wherein said pH of said sodium-ion-containing solution is adjusted to equal to or less than about 4.8 for strong nucleic acid binding.

7. The method of claim 1, wherein said sodium-ion-containing solution is a sodium-acetate solution with sodium acetate concentration of about 2.0 M to 2.5 M, and pH of about 4.0 to 4.8.

8. The method of claim 1, wherein said silicon-containing matrix is selected from silica, celite, glass powders and the like, in different forms.

9. The method of claim 1, wherein said binding buffer further comprises a water-soluble organic solvent for increased reverse binding of said nucleic acid to said silicon-containing matrix.

10. The method of claim 9, wherein said water-soluble organic solvent is selected from the group consisting of ethanol, isopropanol, methanol, ethyl alcohol, or the like.

11. The method of claim 10, wherein said water-soluble organic solvent is ethanol.

12. The method of claim 9, wherein a final concentration of said water soluble organic solvent is about 20% to 50% (v/v).

13. The method of claim 1, wherein said nucleic acid is selected from the group consisting of DNA, plasmid DNA, RNA, or a hybrid molecule of DNA and RNA.

14. The method of claim 1, wherein said biologic sample is selected from the group consisting of animal or human bodily fluids, tissues, organs, cells, PCR products.

15. A nucleic acid obtained from the method of claim 1, wherein said nucleic acid is free of chaotropes or other toxic chemicals.

16. The nucleic acid of claim 13, wherein said nucleic acid is suitable for gene therapy, genetic vaccination, and other enzymatic reactions selected from the group consisting of PCR, restriction digestion, nick translation, labeling, sequencing and tailing,

17. A kit for isolating and purifying nucleic acid comprising a binding buffer comprising a sodium-ion-containing solution for reversible binding of said nucleic acid to a silicon-containing matrix and an instruction, wherein said sodium-ion-containing solution is chaotropic salts free binding solution, wherein a final sodium ion concentration is greater than about 1 M to saturate, and wherein a pH of said sodium-ion-containing solution is adjusted in the range of about 2.0 to 5.0.

18. A method of isolating and purifying nucleic acid from a biological sample comprising providing a binding buffer comprising a potassium-ion-containing solution for reversible binding of said nucleic acid to a silicon-containing matrix, wherein said potassium-ion-containing solution is chaotropic salts free binding solution, wherein a final potassium ion concentration is at least about 500 mM and wherein a pH of said potassium-ion-containing solution is adjusted in the range of about 4.0 to 5.5.

19. The method of claim 18, wherein said binding buffer further comprises about 20% to 30% (v/v) of a water-soluble organic solvent selected from the group consisting of ethanol, isopropanol, methanol, and others for increased reverse binding of said nucleic acid to said silicon-containing matrix.

20. A kit for isolating and purifying nucleic acid comprising a binding buffer comprising a potassium-ion-containing solution for reversible binding of said nucleic acid to a silicon-containing matrix and an instruction, wherein said potassium-ion-containing solution is chaotropic salts free binding solution, wherein a final sodium ion concentration is at least 500 mM, and wherein a pH of said potassium-ion-containing solution is adjusted in the range of about 4.0 to 5.5
